(19) United States
(12) Patent Application Publication
(10) Pub. No.: US 2007/0191629 A1
(43) Pub. Date: Aug. 16, 2007

(54) Methods for preparing 3-hydroxy-propionate and 1,3-propylene glycol

(76) Inventors: Jing Chen, Lanzhou (CN); Fang Cui, Lanzhou (CN); Jianhua Liu, Lanzhou (CN); Chungu Xia, Lanzhou (CN); Jin Tong, Lanzhou (CN)

Correspondence Address: TROUTMAN SANDERS LLP, 600 PEACHTREE STREET , NE, ATLANTA, GA, 30308, US

(21) Appl. No.: 11/516,171

(22) Filed: Sep. 6, 2006

(30) Foreign Application Priority Data
Feb. 16, 2006 (CN) 200610054660.3

(51) Int. Cl. C07C 67/36

(52) U.S. Cl. 560/206; 560/207

(57) ABSTRACT

The invention disclosed a method for producing 3-hydroxy-propionate and 1,3-propylene glycol by using epoxide as raw material. In the method, a transitional metal cobalt catalyst and a cocatalyst for the hydroesterification reaction among epoxide, carbon monoxide, and alcohol were chosen, and a hydrogenation catalyst for hydrogenating 3-hydroxy-propionate as well as the corresponding process conditions were selected for producing 3-hydroxy-propionate and 1,3-propylene glycol.

### TECHNICAL FIELD

The present invention relates to a method for preparing 3-hydroxy-propionate and 1,3-propylene glycol, and more specifically, to methods for preparing 3-hydroxy-propionate and 1,3-propylene glycol by using ethylene oxide as raw material and adopting a catalyst and process conditions suitable for hydroesterification and hydrogenation reaction, respectively.

### BACKGROUND OF THE PRIOR ART

1,3-Propylene glycol(i.e., 1,3-PDO) is a colorless and tasteless viscous liquid, which is soluble in water and a variety of organic solvents such as alcohols, ethers and the like and which can be used mainly to the synthesis of plasticizer, detergent, preservative, and emulsifier, and in the fields of foods, cosmetics, pharmaceuticals and the like. The most important use of 1,3-PDO is to act as a monomer used for synthesizing superior polymer materials, such as the substitutes for ethylene glycol or butylene glycol to produce polyol polyester. In mid 1990s, a novel superior polyester material—polypropylene terephthalate (simply called PTT or 3GT fiber) was successfully synthesized by using 1,3-propylene glycol as raw material. This fiber has the characteristics of polyester and polyamide fiber, as well as the distortional restorability of polyamide fiber, the properties of which are superior compared to those of the polyester fiber material currently produced by using ethylene glycol or 1,4-butylene glycol. The fiber possesses both the high quality of polyethylene terephthalate (i.e., PET) and the easy processing properties of polybutylene terephthalate (i.e., PBT) and has become one of the most promising novel materials in the late 1990s.

In addition, 1,3-propylene glycol can be used to produce other saturated polyesters such as polyethylene naphthalate (PTN), and copolyesters, and to produce novel polyurethanes and fine chemicals, including novel polyurethanes, such as foaming products, elastomers, adhesives, and paints, and fine chemicals, such as antifreezing fluid, powder paints, solvents, road snowbroth agents, medicines and the like.

1,3-Propylene glycol is non-corrosive, easy to biodegrade, and environment friendly. Currently, it is becoming a highly exploitable polymer material monomer.

As a main raw material for synthesizing PDO, ethylene oxide is cheap and plentiful. Therefore, ethylene oxide is used as raw materials to react with synthesis gas to produce 1,3-propylene glycol. One-step and two-step methods was used by Shell company to produce 1,3-propylene glycol. In the methods, ethylene oxide was used to produce 3-hydroxyl-propionaldehyde through hydroformylation reaction followed by hydrogenating (see U.S. Pat. Nos. 5,770,776, 6,180,838, etc). These methods have made improvements to catalysts and processes, however, since the 3-hydroxyl-propionaldehyde itself is an extremely unstable intermediate, the technique for catalytic separation is complicated, which needs to adopt a high pressure reactor with a pressure of higher than 10 MPa. Therefore, the demand for the equipments is strict; the techniques needed are sophisticated; and the cost is high.

In 1990, William (U.S. Pat. No. 4,973,741) produced methyl β-hydroxy-propionate by the carbonylating ethylene oxide. Noble metal-rhodium catalyst and triphenyl phosphine ligand were used in the method under a pressure of 14.0 MPa. It is a method for synthesizing a bifunctional compound, with low conversion rate and low selectivity for desired product.

In the U.S. Pat. No. 6,191,321 filed in 2001 by US Shell Oil company, a method is provided for preparing 1,3-propylene glycol by the hydroesterification reaction of ethylene oxide to synthesize methyl 3-hydroxyl-propionate, followed by hydrogenation. Using Co₂(CO)₈/1,10-phenanthroline as catalyst, methyl tert-butyl ether as solvent, the reaction is carried out at 90° C. under a pressure of 1125 psi for 18 hours. The conversion rate of ethylene oxide is only 11%, and the selectivity of the target product methyl 3-hydroxyl-propionate is 74%. Moreover, the yield of the hydrogenation product is low, and therefore there is little need to make further investment. Korea Samsung Electronic Co Ltd use a binary catalyst system in which Co is use as main catalyst and a nitrogen-containing heterocyclic compound is used as a promoter. In its U.S. Pat. No. 6,521,801, the conversion rate of ethylene oxide reached up to 94% under a carbon monoxide pressure of 6 PMa and a reaction temperature of 75° C., and the selectivity of the desired product 3-hydroxyl-propionate is 78%. In the CN Patent No. 01125121.2 filed by Lanzhou Chemicophysics Research Academy, CSA, a ternary catalyst is disclosed for hydroesterification reaction of propylene oxide, where the yield of methyl β-hydroxyl butyrate is up to 93.2%, and the selectivity is above 97%. The reaction was carried out under an intermediate pressure and the reactor used was a single vessel type.

In the U.S. Pat. No. 6,348,632 of the Korea Samsung Electronic Co Ltd, copper chromate is used as catalyst; 1 gram of methyl 3-hydroxyl-propionate, and 0.5 grams of copper chromate are added into a 45 mL reaction vessel; and the reaction is carried out under a hydrogen pressure of 1500 psi and a temperature of 180° C., where the conversion rate of methyl 3-hydroxyl-propionate is only 5%, and the selectivity of 1,3-propylene glycol is only 3%. In the U.S. Pat. No. 6,617,478, CuO(77% by weight)-SiO₂(20% by weight)-MnO₂(3% by weight) was used as catalyst, and methyl 3-hydroxyl-propionate was hydrogenated into 1,3-propylene glycol. However, it needs 44 hours for the activation of the catalyst. The reaction period lasted for 20 hours under a hydrogen pressure of 1500 psi with a reaction temperature of 150° C. The conversion rate of methyl 3-hydroxyl-propionate is 90.92% and the selectivity of 1,3-propylene glycol is 100%.

### DISCLOSURE OF THE INVENTION

The present invention uses epoxide as raw materials to produce ester intermediate 3-hydroxy-propionate via the hydroesterification reaction among carbon monoxide, alcohol, and epoxide. The resultant ester intermediate was further subjected to a hydrogenation reaction to produce 1,3-propylene glycol. Using ethylene oxide as an example, the reaction principle thereof is shown as follows:

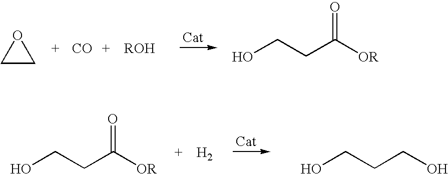

wherein, R is a methyl or ethyl, and the raw material epoxide can also be propylene oxide.

The technical problem sought to be solved by the present invention is to select catalysts suitable for hydroesterification and hydrogenation with high reactivity and selectivity, as well as the corresponding process conditions to produce 3-hydroxy-propionate and 1,3-propylene glycol, respectively.

3-hydroxy-propionate and 1,3-propylene glycol were produced by the present invention by using a transitional metal cobalt catalyst and a cocatalyst in the hydroesterification reaction, and a hydrogenation catalyst in the hydrogenation reaction, as well as employing corresponding reaction conditions. The details are as follows.

In the invention, the raw material epoxide used in the hydroesterification is ethylene oxide or propylene oxide, and the alcohol used is methanol, ethanol, or a mixture thereof. Generally, the reaction can be carried out in an autoclave with the maximum reaction pressure of 20 Mpa. It can also be carried out in a medium pressure reactor (3-8 MPa), or a tubular type reactor with an inner diameter of 20 mm and a length of 1000 mm. This tubular type reactor is connected to a storage tank of carbon monoxide so as to supply the carbon monoxide consumed during the reaction and to keep the reaction system to a certain pressure. The detailed reaction procedure is as follows: The transitional metal cobalt catalyst, in solid or liquid form, and an organic and/or inorganic aid are added into a reaction vessel, then 250-500 ml of alcohol solution is added. The vessel is purged with nitrogen gas for 3 times, and then charged with carbon monoxide and heated. When the reaction temperature is reached, the epoxide is continuously charged, and carbon monoxide is also continuously supplied. The reaction temperature is controlled at 50-100° C.; the reaction pressure is 5.0-7.0 Mpa; and the reaction time is 3-6 hours. After the reaction becomes stable, the reaction liquid is discharged for separation and chromatography analysis.

The purpose of the separation is to separate the catalyst from the reaction mixture through distillation. Flash distillation, thin film evaporation, or vacuum distillation can be use to separate the catalyst and product 3-hydroxy-propionate.

In the invention, the transitional metal cobalt catalyst used for the hydrogenation esterification reaction among ethylene oxide, carbon monoxide and alcohol is selected from pure dicobalt octacarbonyl, cobalt tetracarbonyl and sodium salt thereof, fatty alcohol solution or acetone solution of tetracarbonyl anion, and cobalt carbonyl solution synthesized in situ; the cocatalyst includes an organic compound promoter selected from pyridine, hydroxyl pyridine, quinoline, isoquinoline, and hydroxyl quinoline which are substituted by methyl or dimethyl, and derivatives thereof; and/or an inorganic compound promoter which is a salt of alkali metal or alkaline earth metal selected from sodium acetate, sodium carbonate, sodium bicarbonate, sodium dihydrogen phosphate, sodium sulfate, sodium chloride, sodium bromide, potassium chloride, potassium bromide, potassium hydrogen phosphate diformate, and lithium chloride; when synthesizing the cobalt carbonyl in situ, CoO, Co₃O₄, or CoCO₃ is used as cobalt source with methanol as solvent.

In the catalytic system where the transitional metal cobalt is predominant, the organic or inorganic cocatalyst can be added alone and/or can be used together with water or hydrogen, wherein the molar ratio of Co₂(CO)₈ to the epoxide is 1:100-1:190, and the reaction is carried out at a temperature of 50-100° C., under a pressure of 3.0-7.0 MPa for a reaction period of 3-5 hours. A temperature of 70 to 75° C. is preferred in order to maintain a stable reaction speed. The pressure of carbon monoxide can also be suitably increased to 8-10 MPa so as to improve the selectivity of the product.

Carbon monoxide is the only gas in the hydroesterification reaction. However, since the addition of a small amount of hydrogen can promote the activation of the catalyst and increase the reaction speed, hydrogen can also be used as an effective promoter in the reaction. Alcohol saturated by hydrogen is used as the reaction solvent. Hydrogen with certain pressure can also be charged into the reaction system, and followed by introducing carbon monoxide gas to carry out the reaction. The hydrogen gas is added with a pressure of 0.1 MPa-1.5 MPa, preferably 0.1-0.5 MPa. The addition of excessive hydrogen gas will result in the production of a small amount of aldehyde generated by hydrogen formylation reaction, thereby lowering the selectivity of the product.

In the invention, by using the reactant alcohol itself as solvent, especially when a single methanol is selected, the yield of the product 3-hydroxy-propionate can be improved under suitable reaction temperature and pressure.

Addition a small amount of water can effectively increase the hydroesterification speed of epoxide. When alcohol is used as the solvent, adding 0.5-3.0% by weight of water can decrease the catalytic induction time, accelerate the reaction progress, inhibit the formation of polymers, and increase the selectivity of the product. Water is used as a promoter in the present invention, as a result, the cost is reduced; the reaction effect is significant; and no harmful substance will be brought into the reaction system.

As cocatalysts, organic aid and inorganic aid have synergistic effect. Using of both together will lead to better result. In the invention, when 3-hydroxyl pyridine which is an organic aid and sodium acetate which is an inorganic aid are used together with a molar ratio of 1:1, the selectivity of the product 3-hydroxy-propionate is higher than 97%.

In the invention, the hydrogenation reaction can hydrogenate the 3-hydroxy-propionate obtained by separating the catalyst from the reaction mixture produced by the hydroesterification reaction. 3-Hydroxy-propionate is directly treated by the hydrogenation catalyst of the invention to produce 1,3-propylene glycol. Intermediate 3-Hydroxy-propionate should be further purified via ordinary vacuum distillation for example. The purity of the product purified can be more than 98%.

The hydrogenation catalyst of the invention consists of a composite oxide of titanium dioxide and silica as carrier and cuprous chloride as an aid.

The hydrogenation catalyst of the invention is prepared by coprecipitation, slurry mixing or immersion. The coprecipitation method is a method of adding a basic precipitant such as sodium hydroxide or the like into a salt solution containing the respective components of the catalyst. The slurry mixing method is a method of precipitating the active component copper salt and the silicon-containing complex, respectively, and then mixing the slurries. The immersion method is a method of immersing the catalyst aid of CuCl on the catalyst.

In one embodiment of the present invention, the catalyst is prepared as follows.

The catalyst of the invention includes a composite oxide of titanium dioxide and silica as carrier. Titanium dioxide and the silica composite oxide account for 55%-80% by weight of the total weight of the catalyst, wherein the ratio of titanium dioxide and the silica composite oxide TiO₂:SiO₂ is 0.5%-50%:50%-99.5% by weight; preferably 8%-20%:80%-92%. Copper oxide is used as an active component, accounting for 15%-40% by weight of the total weight of the catalyst. CuCl is used as an aid, accounting for 0.5%-10% by weight of the total weight of the catalyst.

The Catalyst is Prepared by the Following Steps:

1. formulating the compounds which contain each component of the catalyst into a solution according to the desired components and blending ratio, wherein the total concentration of the compounds is 5-40% by weight, preferably 10%-20% by weight; co-precipitating with sodium hydroxide having a concentration of 5%-20%, preferably 7%-15% by weight, or mixing co-precipitate (A) of a compound containing active component and cocatalyst CuCl with co-precipitate (B) of a titanium and silicon-containing complex, wherein the concentration of sodium hydroxide is 5-20% by weight, preferably 7-15% by weight. The precipitation is carried out at room temperature. The resultant is subjected to aging process at 80° C. for 4 hours, filtrated, washed until the filtrate became neutral, dried at 120° C. for 16 hours, and baked at 600° C. for 4 hours, or the aid can be impregnated at the surface of the catalyst and then baked at 350° C. for 4 hours, to obtain a composite oxide;

2. crushing the above composite oxide into fine particles, and screening to obtain carriers having a size of 20 to 60 mesh, and

3. reducing the above carriers with hydrogen at 300° C. for 4 hours.

In order to increase the activity and selectivity of the hydrogenation catalyst and to avoid side-effects such as reactant being dehydrated, the formation of lactone, the degradation of the ester group or the like caused by high temperature, it is necessary to increase the strength of the catalyst (to increase the content of the catalyst carrier) to meet the requirements of a fixed bed reactor.

The invention provides a method to produce 3-hydroxy-propionate and 1,3-propylene glycol respectively, wherein epoxide was used as raw material, and two catalysts and process conditions suitable for hydrogen esterfication and hydrogenation reaction were chosen. Compared with other methods, the advantages of the present invention are as follows. The reaction equipments are simple, the operation is convenient, and the reaction conditions are wild. The reaction can not only be carried out in a single pot reactor, but in a tubular type reactor. The temperature and the pressure are not so high. The activity of the catalyst is high and the stability is good. The catalyst can be produced easily, and the raw materials thereof are easy to obtain, and the cost is lowered. After product separation, the selectivity of the catalyst can be improved by adding promoters. The intermediate 3-hydroxy-propionate is easy to be purified, and has high yield, selectivity and stability, which can be used as intermediates for fine chemicals, and can be used in various application filed. There is no environmental pollution during the process. The propylene product of the hydrogenation reaction has a high concentration, is easy to be purified by rectification, and has a lower production cost. During the hydroesterification reaction, alcohol is used both as a reactant and a solvent, and a small amount of hydrogen gas and water is introduced into the reaction system, all these are of benefit to improve the product yield and the selectivity. By using the cocatalyst CuCl and the composite oxide, the reactivity of the catalyst and the selectivity has been improved significantly.

### DETAILED DESCRIPTION OF THE PREFERRED EMBODIMENT

Cobalt catalyst (solid or solution), and organic and/or inorganic aids were added into a 1 to 2 L stainless steel autoclave equipped with an electromagnetic stirring apparatus. Then, a certain amount of alcohol solution was added. After the autoclave was purged with nitrogen three times, CO was charged into the autoclave with a certain pressure, followed by heating. After reaching reaction temperature, ethylene oxide was consecutively charged, and CO was supplied continuously. The reaction was carried out for 3 hours-6 hours, under conditions that the temperature was maintained at 50° C.-100° C. and the pressure was maintained at 5.0 MPa-7.0 MPa. Thereafter, the reaction mixture was cooled to room temperature, and the reaction liquid was analyzed.

The Invention is Further Illustrated as Follows.

### EXAMPLE 1

In a 1 L stainless steel autoclave, 450 mL of methanol, 5.0 g of Co₂(CO)₈, 3.5 mL of quinoline, 6 mL of water, and 0.5 MPa of H₂ were added. CO gas with a pressure of 4.0 MPa and 120 g of ethylene oxide were charged at room temperature. The reaction temperature was maintained at 65° C.-75° C., and the reaction pressure was maintained at 5.5 MPa. The reaction was carried out for 6 hours. The content of the methyl 3-hydroxyl-propionate contained in the solution was 16%. The selectivity was 89.5%.

### EXAMPLE 2

450 mL of methanol, 5.0 g of Co₂(CO)₈, 3.5 mL of isoquinoline, 7.0 mL of water, and 0.5 MPa of H₂ were added. CO gas with a pressure of 4.0 MPa and 120 g of ethylene oxide were charged at room temperature. The reaction temperature was maintained at 65° C.-75° C., and the reaction pressure was maintained at 5.5 MPa. The reaction was carried out for 6 hours. The selectivity of methyl 3-hydroxyl-propionate was 88%, that of methyl acrylate was 7.2%, that of ethylene glycol monomethyl ether was 0.5%, and those of others were 2.0%.

### EXAMPLE 3

450 mL of methanol, 5.0 g of Co₂(CO)₈, 4.2 g of 8-hydroxyquinoline, and 0.5 MPa H₂ were added. CO gas with a pressure of 4.0 MPa and 120 g of ethylene oxide were charged at room temperature. The reaction temperature was maintained at 65° C.-75° C., and the reaction pressure was maintained at 5.5 MPa. The reaction was carried out for 6 hours. There was a small amount of precipitate of about 1.9 g in the reaction mixture liquid. The selectivity of methyl 3-hydroxyl-propionate was 60.7%, that of methyl acrylate was 16.6%, that of ethylene glycol monomethyl ether was 5.9%, and those of others were 16.8%.

### EXAMPLE 4

250 mL of methanol, 5.0 g of Co₂(CO)₈, 5.5 g of 3-hydroxyl pyridine, 5.0 mL of water, and 1.0 MPa of H₂ were added. CO gas with a pressure of 4.0 MPa and 120 g of ethylene oxide were charged at room temperature. The reaction temperature was maintained at 80° C.-85° C., and the reaction pressure was maintained at 5.5 MPa. The reaction was carried out for 6 hours. The selectivity of methyl 3-hydroxyl-propionate was 75.6%, and that of acetaldehyde was 24.4%.

### EXAMPLE 5

250 mL of methanol, 5.0 g of Co₂(CO)₈, 5.5 g of 3-hydroxyl pyridine, 2.5 mL of water, and 0.5 MPa of H₂ were added. CO gas with a pressure of 4.0 MPa and 120 g of ethylene oxide were charged at room temperature. The reaction temperature was maintained at 75° C.-80° C., and the reaction pressure was maintained at 5.5 MPa. The reaction was carried out for 4 hours. The selectivity of methyl 3-hydroxyl-propionate 83.2%, that of acetaldehyde was 6.8%, that of oligomer was 6.1%, that of methyl acrylate was 2.7%, and that of ethylene glycol monomethyl ether was 1.1%.

### EXAMPLE 6

400 mL of methanol, 5.0 g of Co₂(CO)₈, 5.5 g of 3-hydroxyl pyridine, 4.0 mL of water, and 0.5 MPa of H₂ were added. CO gas with a pressure of 4.0 MPa and 120 g of ethylene oxide were charged at room temperature. The reaction temperature was maintained at 75° C.-80° C., and the reaction pressure was maintained at 5.5 MPa. The reaction was carried out for 4 hours. The selectivity of methyl 3-hydroxyl-propionate 96.2%, that of acetaldehyde was 1.5%, that of methyl acrylate was 2.4%, and that of oligomer was trace amount. The reaction liquid was separated under vacuum, leaving the catalyst in the autoclave, and 400 mL of methanol, 4.0 mL of water, and 120 g of ethylene oxide were further added. The reaction was carried out under the above conditions for 4 hours. Both reaction liquids were combined and rectified under vacuum to obtain 380 g of methyl 3-hydroxyl-propionate with a purity of 96.5%.

### EXAMPLE 7

500 mL of methanol, 5.0 g of Co₂(CO)₈, 5.5 g of 3-hydroxyl pyridine, and 4.0 mL of water were added. CO gas with a pressure of 4.5 MPa and 120 g of ethylene oxide were charged at room temperature. The reaction temperature was maintained at 74° C.-76° C. The reaction was carried out for 4 hours. The selectivity of methyl 3-hydroxyl-propionate was above 98%.

### EXAMPLE 8

450 mL of methanol, 5.0 g of Co₂(CO)₈, 5.5 g of 3-hydroxyl pyridine, 4.5 mL of water, and 0.5 MPa of H₂ were added. CO gas with a pressure of 4.0 MPa and 120 g of ethylene oxide were charged at room temperature. The reaction temperature was maintained at 75° C.-80° C., and the reaction pressure was maintained at 4.5 MPa-5.0 MPa. The reaction was carried out for 4 hours. The reaction liquid was distilled to obtain 186 g of methyl 3-hydroxyl-propionate with a purity of 94%. The selectivity of methyl 3-hydroxyl-propionate was 93.6%, that of acetaldehyde was 1.9%, that of oligomer was 1.1%, that of methyl acrylate was 3.0%, and that of ethylene glycol monomethyl ether was 0.5%.

### EXAMPLE 9

400 mL of methanol, 5.0 g of Co₂(CO)₈, and 3.0 g of 3-hydroxyl pyridine were added. CO gas with a pressure of 4.0 MPa and 120 g of ethylene oxide were charged at room temperature. The reaction temperature was maintained at 75° C.-80° C., and the reaction pressure was maintained at 4.5 MPa-5.0 MPa. The reaction was carried out for 4 hours. The selectivity of methyl 3-hydroxyl-propionate was 90.3%, that of acetaldehyde was 2.3%, that of oligomer was 2.1%, that of methyl acrylate was 3.8%, and that of ethylene glycol monomethyl ether was 1.5%.

### EXAMPLE 10

450 mL of methanol, 5.0 g of Co₂(CO)₈, 5.5 g of 3-hydroxyl pyridine, 3.0 mL of water, 0.5 MPa of H₂ were added. CO gas with a pressure of 4.0 MPa and 120 g of ethylene oxide were charged at room temperature. The reaction temperature was maintained at 75° C.-80° C., and the reaction pressure was maintained at 4.5 MPa-5.0 MPa. The reaction was carried out for 4 hours. The reaction liquid was distilled to obtain 191 g of methyl 3-hydroxyl-propionate with a purity of 92.9%. The selectivity of methyl 3-hydroxyl-propionate was 95.4%, that of acetaldehyde was 1.9%, and that of methyl acrylate was 2.7%.

### EXAMPLE 11

450 ml of industrial methanol, 5.0 g of Co₂(CO)₈, 5.5 g of 3-hydroxyl pyridine, 7.0 mL of water, and 0.5 MPa of H₂ were added. CO gas with a pressure of 4.0 MPa and 120 g of ethylene oxide were charged at room temperature. The reaction temperature was maintained at 75° C.-80° C., and the reaction pressure was maintained at 4.5 MPa-5.0 MPa. The reaction was carried out for 4 hours. The selectivity of methyl 3-hydroxyl-propionate was 94.6%, that of acetaldehyde was 2.2%, that of methyl acrylate was 3.2%. The reaction liquid was distilled to obtain 183 g of methyl 3-hydroxyl-propionate with a purity of 92.1%.

### EXAMPLE 12

Into a 35 ml autoclave, 4 ml of methanol, 1 ml of propylene, 0.043 g of Co₂(CO)₈, and 0.035 g of K₂CO₃ were added. CO gas was charged at room temperature. The reaction was carried out at 80° C. under a reaction pressure being controlled at 6.0 MPa for 10 hours. The selectivity of methyl 3-hydroxyl-butyrate was 96.4%.

### EXAMPLE 13

In the same manner as that in Example 12 except that the K₂CO₃ was changed to 0.026 g of Na₂CO₃. The selectivity of methyl 3-hydroxyl-butyrate was 96.3%.

### EXAMPLE 14

In the same manner as that in Example 12 except that the K₂CO₃ was changed to 0.024 g of 3-hydroxyl pyridine. The selectivity of methyl 3-hydroxyl-butyrate was 93.4%.

### EXAMPLE 15

In the same manner as that in Example 12 except that the K₂CO₃ was changed to 0.035 g of K₂CO₃ and 0.024 g of 3-hydroxyl pyridine. The selectivity of methyl 3-hydroxyl-butyrate was 93.5%.

### EXAMPLE 16

In 100 ml of distilled water, 20 grams of Cu(NO₃)₂.3H₂O; 1.5 grams of CuCl; 26.0 grams of Na₂SiO₃.9H₂O and 2.3 ml of ethanol solution containing 30% of TiCl₄ were added. They were coprecipitated at PH=8 with 10% of NaOH at room temperature; aged at 80° C. for 4 hours, filtrated, and washed until the filtrate was neutral, dried at 120° C. for 16 hours, baked at 600° C. for 4 hours, and crushed into fine particles, and screened. The particles with a particle size of 20-40 mesh were selected.

### EXAMPLE 17

20 grams of Cu(NO₃)₂.3H₂O and 1.5 grams of CuCl were dissolved in 40 ml of distilled water. The mixture was precipitated at PH=8 with 10% sodium hydroxide to obtain liquid A. 26.0 grams of Na₂SiO₃.9H₂O and 2.3 ml of ethanol solution containing 30% of TiCl₄ were dissolved in 60 ml of distilled water. The mixture was precipitated at PH=8 with 10% sodium hydroxide at room temperature to obtain liquid B. The liquid A and liquid B were mixed, aged at 80° C. for 4 hours, filtrated, and washed until the filtrate was neutral, dried at 120° C. for 16 hours, baked at 600° C. for 4 hours, and crushed into fine particles, and screened. The particles with a particle size of 20-40 mesh were selected.

### EXAMPLE 18

20 grams of Cu(NO₃)₂.3H₂O was dissolved in 40 ml of distilled water, and precipitated at PH=8 with 10% by weight of sodium hydroxide to obtain liquid A. 26.0 grams of Na₂SiO₃.9H₂O and 2.3 ml of ethanol solution containing 30% of TiCl₄ were dissolved in 60 ml of distilled water, and then precipitated at PH=8 with 10% by weight of sodium hydroxide to obtain liquid B. The liquid A and liquid B were mixed, aged at 80° C. for 4 hours, filtrated, and washed until the filtrate became neutral, dried at 120° C. for 16 hours, baked at 600° C. for 4 hours, and crushed into fine particles, and screened. The particles with a particle size of 20-40 mesh were selected for future use. 1.0 gram of CuCl was prepared into 20 ml of solution, and the above catalyst particles were immersed into the solution for 8 hours, and baked at 350° C. for 4 hours. The catalysts prepared by the above three methods were charged into fixed reactors each having an inner diameter of 15 mm and a length of 250 mm, respectively. The charging amount of the catalyst was 4 grams. H₂ was firstly introduced into the reactor, and then the temperature was increased to 300° C. with a increasing speed of 0.5° C./min. The resultant was reduced in situ for 4 hours, followed by decreasing the temperature to 140° C. A methanol solution containing methyl 3-hydroxyl-propionate was introduced into the reactor to carry out a hydrogenation reaction and the results are shown in table 1.

TABLE 1

| | The selectivity of methyl | The selectivity of |
|---|---|---|
| Catalyst | 3-hydroxyl-propionate % | 1,3-propylene glycol % |
| Method 1 | 80.3 | 79.6 |
| Method 2 | 90.9 | 87.1 |
| Method 3 | 93.9 | 88.2 |

What is claimed is:

1. A method for producing 3-hydroxy-propionate, comprising the steps of:
providing an epoxide as raw material;
providing a transitional metal cobalt catalyst and a cocatalyst; and
reacting the epoxide, a carbon monoxide, and an alcohol in the presence of the catalyst and the cocatalyst under appropriate reaction conditions to produce 3-hydroxy-propionate (Reaction 1);
wherein, the transitional metal cobalt catalyst includes a pure dicobalt octacarbonyl, cobalt tetracarbonyl and sodium salt thereof, a fatty alcohol solution or acetone solution of a tetracarbonyl anion, and a solution of cobalt carbonyl synthesized in situ, and
the cocatalyst includes an organic compound promoter including pyridine, hydroxyl pyridine, quinoline, isoquinoline, and hydroxyl quinoline substituted by methyl or dimethyl, and derivatives thereof, and/or a inorganic compound promoter which is a salt of alkali metal or alkaline earth metal including sodium acetate, sodium carbonate, sodium bicarbonate, sodium dihydrogen phosphate, sodium sulfate, sodium chloride, sodium bromide, potassium chloride, potassium bromide, potassium hydrogen phosphate diformate, and lithium chloride.

2. A method for producing 1,3-propylene glycol, comprising the steps of:
providing an epoxide as raw material;
providing a transitional metal cobalt catalyst and a cocatalyst;
reacting the epoxide, a carbon monoxide, and an alcohol in the presence of the catalyst and the cocatalyst under appropriate reaction conditions to produce 3-hydroxy-propionate (Reaction 1);
providing a hydrogenation catalyst;
providing hydrogen gas; and
reacting the hydrogen gas with the 3-hydroxy-propionate in the presence the hydrogenation catalyst under appropriate reaction conditions to produce 1,3-propylene glycol (Reaction 2);
wherein, the transitional metal cobalt catalyst includes a pure dicobalt octacarbonyl, cobalt tetracarbonyl and sodium salt thereof, a fatty alcohol solution or acetone solution of a tetracarbonyl anion, and a solution of cobalt carbonyl synthesized in situ,
the cocatalyst includes an organic compound promoter including pyridine, hydroxyl pyridine, quinoline, isoquinoline, and hydroxyl quinoline substituted by methyl or dimethyl, and derivatives thereof, and/or a inorganic compound promoter which is a salt of alkali metal or alkaline earth metal including sodium acetate, sodium carbonate, sodium bicarbonate, sodium dihydrogen phosphate, sodium sulfate, sodium chloride, sodium bromide, potassium chloride, potassium bromide, potassium hydrogen phosphate diformate, and lithium chloride, and
the hydrogenation catalyst consists of a composite oxide of titanium dioxide and silica as a carrier and cuprous chloride as an aid.

3. A method for producing 3-hydroxy-propionate by using an epoxide as raw material according to claim 1, wherein
in the catalyst system in which the transitional metal cobalt is predominant, the organic compound promoter and/or the inorganic compound promoter can be added alone and/or can be used together with water or hydrogen, and wherein
the molar ratio of dicobalt octacarbonyl to the epoxide is 1:100-1:190, and the reaction is carried out at a temperature of 50-100° C., under a pressure of 0-7.0 MPa for a reaction period of 3-5 hours.

4. A method for producing 1,3-propylene glycol by using an epoxide as raw material according to claim 2, wherein
in the catalyst system in which the transitional metal cobalt is predominant, the organic compound promoter and/or the inorganic compound promoter can be added alone and/or can be used together with water or hydrogen, and wherein
the molar ratio of dicobalt octacarbonyl to the epoxide is 1:100-1:190, and the reaction is carried out at a temperature of 50-100° C., under a pressure of 0-7.0 MPa for a reaction period of 3-5 hours.

5. A method for producing 3-hydroxy-propionate by using an epoxide as raw material according to claim 3, wherein
the temperature is 70-75° C.

6. A method for producing 1,3-propylene glycol by using an epoxide as raw material according to claim 4, wherein
the temperature is 70-75° C.

7. A method for producing 3-hydroxy-propionate by using an epoxide as raw material according to claim 1, wherein
the epoxide includes ethylene oxide and propylene oxide, and the alcohol includes methanol, ethanol or the mixture thereof.

8. A method for producing 1,3-propylene glycol by using an epoxide as raw material according to claim 2, wherein
the epoxide includes ethylene oxide and propylene oxide, and the alcohol includes methanol, ethanol or the mixture thereof.

9. A method for producing 3-hydroxy-propionate by using an epoxide as raw material according to claim 1, wherein
in reaction 1, an alcohol saturated by hydrogen is used as a reaction solvent, and hydrogen having a pressure of 0.1-1.5 MPa is added as a promoter to accelerate the activation of the catalysts.

10. A method for producing 1,3-propylene glycol by using an epoxide as raw material according to claim 2, wherein
in reaction 1, an alcohol saturated by hydrogen is used as a reaction solvent, and hydrogen having a pressure of 0.1-1.5 MPa is added as a promoter to accelerate the activation of the catalysts.

11. A method for producing 3-hydroxy-propionate by using an epoxide as raw material according to claim 9, wherein
in reaction 1, the pressure of the hydrogen added is 0.1-0.5 MPa.

12. A method for producing 1,3-propylene glycol by using an epoxide as raw material according to claim 10, wherein
in reaction 1, the pressure of the hydrogen added is 0.1-0.5 MPa.

13. A method for producing 3-hydroxy-propionate by using an epoxide as raw material according to claim 1, wherein
when an alcohol is used as the solvent in the reaction 1, water is added in an amount of 0.5-3% by weight as the promoter.

14. A method for producing 1,3-propylene glycol by using an epoxide as raw material according to claim 2, wherein
when an alcohol is used as the solvent in the reaction 1, water is added in an amount of 0.5-3% by weight as the promoter.

15. A method for producing 3-hydroxy-propionate by using an epoxide as raw material according to claim 1, wherein
3-hydroxyl pyridine as an organic compound promoter and sodium acetate as an inorganic compound promoter are used together, with the molar ratio of 3-hydroxyl pyridine to sodium acetate of 1:1.

16. A method for producing 1,3-propylene glycol by using an epoxide as raw material according to claim 2, wherein
3-hydroxyl pyridine as an organic compound promoter and sodium acetate as an inorganic compound promoter are used together, with the molar ratio of 3-hydroxyl pyridine to sodium acetate of 1:1.

17. A method for producing 3-hydroxy-propionate by using an epoxide as raw material according to claim 1, wherein
the cobalt carbonyl synthesized in situ is produced by using CoO, Co₃O₄, or CoCO₃ as a cobalt source and using methanol as a solvent.

18. A method for producing 1,3-propylene glycol by using an epoxide as raw material according to claim 2, wherein
the cobalt carbonyl synthesized in situ is produced by using CoO, Co₃O₄, or CoCO₃ as a cobalt source and using methanol as a solvent.

19. A method for producing 1,3-propylene glycol by using an epoxide as raw material according to claim 2, wherein
the composite oxide accounts for 55-80% by weight of the catalyst, the weight ratio of TiO₂:SiO₂ in the composite oxide being 0.5%-50%: 50%-99.5%, active ingredient cupper oxide being 15-40% by weight of the catalyst, and the cuprous chloride being 0.5-10% by weight of the catalyst.

20. A method for producing 1,3-propylene glycol by using an epoxide as raw material according to claim 19, wherein
TiO₂:SiO₂=8%-20%:80%-92%.

21. A method for producing 1,3-propylene glycol by using an epoxide as raw material according to claim 2, wherein
the hydrogenation catalyst is produced by:
formulating compounds which contain each component of the catalyst into a solution according to the desired components and blending ratio, wherein the total concentration of the compounds is 5-40% by weight, and then co-precipitating with sodium hydroxide having a concentration of 5%-20%; or
mixing a first co-precipitate of a compound containing active component and cocatalyst CuCl with a second co-precipitate of a titanium and silicon-containing complex, the concentration of sodium hydroxide being 5-20% by weight, wherein the precipitation is carried out at room temperature, and the resultant is subjected to aging process at 80° C. for 4 hours, filtrated, washed until the filtrate becomes neutral, dried at 120° C. for 16 hours, and baked at 600° C. for 4 hours; or the aid can be impregnated at the surface of the catalyst and then baked at 350° C. for 4 hours to obtain a composite oxide;
crushing the composite oxide, and screening to obtain a carrier having a size of 20 to 60 mesh; and
reducing the carrier with hydrogen at 300° C. for 4 hours.

22. A method for producing 1,3-propylene glycol by using an epoxide as raw material according to claim 19, wherein
the hydrogenation catalyst is produced by:
formulating compounds which contain each component of the catalyst into a solution according to the desired components and blending ratio, wherein the total concentration of the compounds is 5-40% by weight, and then co-precipitating with sodium hydroxide having a concentration of 5%-20%; or
mixing a first co-precipitate of a compound containing active component and cocatalyst CuCl with a second co-precipitate of a titanium and silicon-containing complex, the concentration of sodium hydroxide being 5-20% by weight, wherein the precipitation is carried out at room temperature, and the resultant is subjected to aging process at 80° C. for 4 hours, filtrated, washed until the filtrate becomes neutral, dried at 120° C. for 16 hours, and baked at 600° C. for 4 hours; or the aid can be impregnated at the surface of the catalyst and then baked at 350° C. for 4 hours to obtain a composite oxide;
crushing the composite oxide, and screening to obtain a carrier having a size of 20 to 60 mesh; and
reducing the carrier with hydrogen at 300° C. for 4 hours.

23. A method for producing 1,3-propylene glycol by using an epoxide as raw material according to claim 20, wherein
the hydrogenation catalyst is produced by:
formulating compounds which contain each component of the catalyst into a solution according to the desired components and blending ratio, wherein the total concentration of the compounds is 5-40% by weight, and then co-precipitating with sodium hydroxide having a concentration of 5%-20%; or
mixing a first co-precipitate of a compound containing active component and cocatalyst CuCl with a second co-precipitate of a titanium and silicon-containing complex, the concentration of sodium hydroxide being 5-20% by weight, wherein the precipitation is carried out at room temperature, and the resultant is subjected to aging process at 80° C. for 4 hours, filtrated, washed until the filtrate becomes neutral, dried at 120° C. for 16 hours, and baked at 600° C. for 4 hours; or the aid can be impregnated at the surface of the catalyst and then baked at 350° C. for 4 hours to obtain a composite oxide;
crushing the composite oxide, and screening to obtain a carrier having a size of 20 to 60 mesh; and
reducing the carrier with hydrogen at 300° C. for 4 hours.

24. A method for producing 1,3-propylene glycol by using an epoxide as raw material according to claim 21, wherein
the total concentration of the compounds is 10-20% by weight of the solution, and the sodium hydroxide has a concentration of 7%-15% by weight.

25. A method for producing 1,3-propylene glycol by using an epoxide as raw material according to claim 22, wherein
the total concentration of the compounds is 10-20% by weight of the solution, and the sodium hydroxide has a concentration of 7%-15% by weight.

26. A method for producing 1,3-propylene glycol by using an epoxide as raw material according to claim 23, wherein
the total concentration of the compounds is 10-20% by weight of the solution, and the sodium hydroxide has a concentration of 7%-15% by weight.
